(19) United States
(12) Patent Application Publication
(10) Pub. No.: US 2024/0254548 A1
(43) Pub. Date: Aug. 1, 2024

(54) BOOTSTRAPPING A DNA DATA STORAGE ARCHIVE

(71) Applicant: MICROSOFT TECHNOLOGY LICENSING, LLC, Redmond, WA (US)

(72) Inventors: Karin STRAUSS, Seattle, WA (US); Patrick L. GARVAN, Monroe, WA (US)

(21) Appl. No.: 18/160,988

(22) Filed: Jan. 27, 2023

(51) Int. Cl. C12Q 1/6869; C12Q 1/686; G06N 3/123; G16B 30/20; G16B 50/30

(52) U.S. Cl. CPC C12Q 1/6869; C12Q 1/686; G06N 3/123; G16B 30/20; G16B 50/30; C12Q 2600/16

(57) ABSTRACT

This disclosure describes a technique for bootstrapping the reading of a DNA data storage archive from information contained in the oligonucleotides of the archive. Labeling oligonucleotides are added to the DNA data storage archive. The labeling oligonucleotides are amplified by a known pair of primers. The sequence of nucleotides between the primers functions as an uncoded identifier that is used to look up a decoding technique. The association between the uncoded identifier and the decoding technique may be stored in a network-accessible database. Data storage oligonucleotides in the DNA data storage archive can then be decoded and digital data recovered by use of the decoding technique. Knowledge of the primers and location of the database is thus sufficient to read the DNA data storage archive even if external labeling information that provides the decoding technique for the archive is lost.

### SEQUENCE LISTING

The Sequence Listing associated with this application is provided as a Sequence Listing XML in accordance with WIPO Standard ST.26 and is hereby incorporated by reference into the specification. The name of the XML file containing the Sequence Listing is MS1-9803US_sequence_ST26.xml. The file is 5 kb, was created on Jan. 18, 2023, and is being submitted electronically concurrent with the filing of the specification.

### BACKGROUND

Reading digital data stored in deoxyribose nucleic acid (DNA), or other oligonucleotides, requires knowledge of the decoding technique used to convert the nucleotide sequences into a string of zeros and ones. Typically, the required decoding techniques and parameters are recorded in an externally-persisted record such as a laboratory notebook or electronic database. For convenience, an indication of the appropriate decoding technique is often attached to a physical container that holds the oligonucleotides of a DNA data storage archive or simply “archive.” For example, the name of the decoding technique may be written on the outside of a tube that holds the DNA. A person wishing to recover digital data from the DNA can then look up the details of the decoding technique associated with that name.

However, the label may be lost or become unreadable. The oligonucleotides may be transferred to a different container that is not labeled or mislabeled. The meaning of labeling nomenclature that is unique to a particular individual or organization may be undecipherable to others. Thus, it may become impossible to decode information in a DNA data storage archive even though there is no problem with the DNA itself. This may be a significant problem with archives that are maintained for many tens or hundreds of years.

It would be useful to have some way of decoding an otherwise usable DNA data storage archive even if the external labeling information is lost or unreadable. The following disclosure is made with respect to these and other considerations.

### SUMMARY

This disclosure describes bootstrapping a DNA data storage archive to obtain a decoding technique for the archive. This can be done in the absence of external labeling information. Bootstrapping uses only a minimal set of starting information to identify the proper technique for decoding the DNA. Specifically, a sequence of a primer pair that are used to amplify labeling oligonucleotides and the location of a look-up table that contains the decoding technique are all that is needed.

The primer pair is used for polymerase chain reaction (PCR) amplification of labeling oligonucleotides included in the DNA data storage archive. Labeling oligonucleotides are special oligonucleotides mixed in with data-encoding oligonucleotides. After PCR amplification, there are a much larger number of labeling oligonucleotides than other nucleotides in the archive. Thus, sequencing the contents of the archive provides the sequence of the labeling oligonucleotides. This sequence, a string of As, Gs, Cs, and Ts, is used directly as an index to query a look-up table. The look-up table contains many nucleotide sequences and each is associated or linked with a decoding technique. Each decoding technique provides the information necessary to decode the data storage oligonucleotides in the archive. This information may include identification of primers for amplifying the data-encoding nucleotides and an algorithm for converting a nucleotide sequence to digital data.

The look-up table may be maintained by a central authority as a public specification or standard. For example, the look-up table could be publicly available on the Internet. All entities that store digital data in DNA data storage archives may be aware of this standard. DNA data storage archives that conform to this standard include labeling oligonucleotides that are amplified by the same primer pair. The sequence of this primer pair will be publicly known and published as part of the standard. Thus, any DNA data storage archive that conforms to this standard can be decoded even if all external labeling information (that would normally provide the decoding technique) is lost.

One variation of this technique uses two different sets of labeling oligonucleotides to provide greater flexibility and specificity. The sequence of the first set of labeling oligonucleotides is used to query a look-up table and identify a decoding technique as described above. That decoding technique is then used to decode a second set of labeling oligonucleotides that are also included in the archive. Thus, the archive includes first labeling oligonucleotides, second labeling oligonucleotides, and data storage oligonucleotides. The second labeling oligonucleotides, once decoded with the decoding technique provided by the first labeling oligonucleotides, can provide any information that would be included in a physical label on the archive. Typically, the second labeling oligonucleotides will contain a second decoding technique that is used to decode the data-encoding polynucleotides.

Creating the look-up table involves making associations between nucleotide sequences and decoding techniques. As a publicly available standard that could be implemented for all DNA data storage archives, there may be a large number of different decoding techniques included. Each is identified by a unique nucleotide sequence. Over time additional decoding techniques can be added to the look-up table. Thus, there is a mechanism to assign nucleotide sequences to decoding techniques.

Because the nucleotide sequences used to query the look-up table come from reads of labeling oligonucleotides there may be errors. The errors could be introduced from the synthesis or sequencing of the labeling oligonucleotides. If there are errors, the sequence reported from a labeling oligonucleotide may not be an exact match to any of the nucleotide sequences in the look-up table. Thus, the technique for querying the look-up table accommodates approximate matching such as, for example, by finding a match with a minimum edit distance.

In order to reduce ambiguity when making approximate matches, the nucleotide sequences assigned to the decoding technique are selected to ensure a certain minimum level of dis-similarity. For example, if two nucleotide sequences in the look-up table differ only in a single nucleotide (e.g., AAATG vs. AACTG) it may be difficult to determine which is the best approximate match for a query that is similar to both (e.g., for AAGTG). Thus, each nucleotide sequence used in the look-up table as an identifier is selected so that it is at least a minimum edit distance from all other nucleotide sequences. This creates sufficient difference between the nucleotide sequences so a query of the look-up table with an approximate match will resolve with high confidence to a single decoding technique.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures are schematic representations and items shown in the figures are not necessarily to scale.

FIGS. 1A and 1B show the contents of a DNA data storage archive that contains labeling oligonucleotides and the use of a look-up table to identify a decoding technique for decoding data storage oligonucleotides to obtain digital data.

FIGS. 2A and 2B show the contents of a DNA data storage archive that contains labeling oligonucleotides and second labeling oligonucleotides. FIGS. 2A and 2B also show the use of a look-up table to identify a decoding technique for decoding the second labeling oligonucleotides which provides a decoding technique to decode data storage oligonucleotides and obtain digital data.

FIG. 3 shows adding a new decoding technique to a look-up table in a database maintained by a central authority and generating a sequence of nucleotides to be an uncoded identifier associated with that decoding technique.

FIG. 4 shows querying a database containing a look-up table with a nucleotide sequence and retrieving a decoding technique from the database.

FIG. 5 is a flow diagram showing an illustrative process for using labeling oligonucleotides to bootstrap identification of a decoding technique to recover digital data from data storage oligonucleotides.

FIG. 6 is a flow diagram showing an illustrative process for adding a decoding technique to a look-up table and assigning an uncoded identifier to the decoding technique.

FIG. 7 is a computer architecture diagram illustrating an illustrative computer hardware and software architecture for a computing system capable of implementing aspects of the techniques and technologies presented herein.

### DETAILED DESCRIPTION

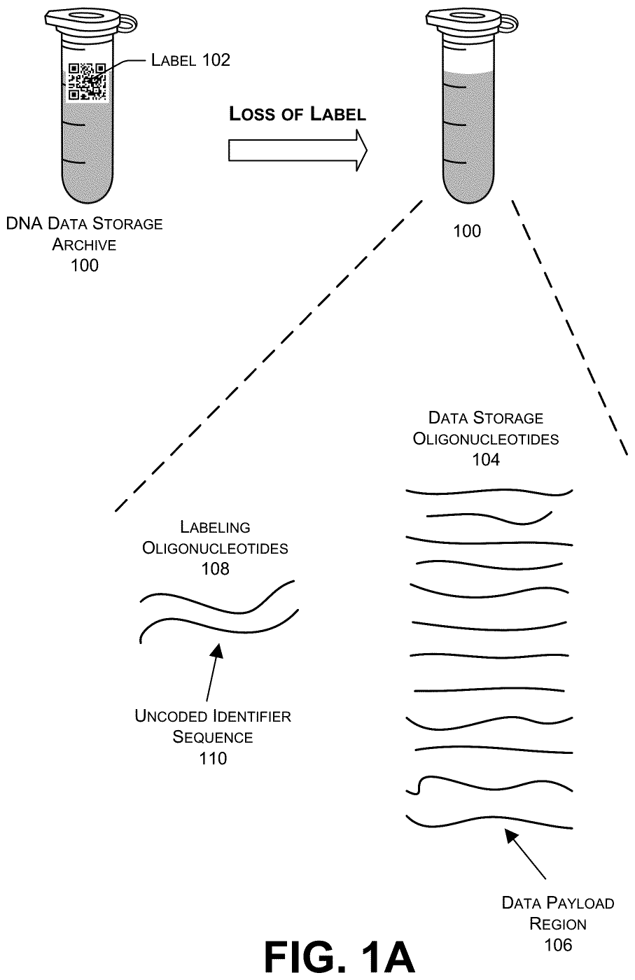

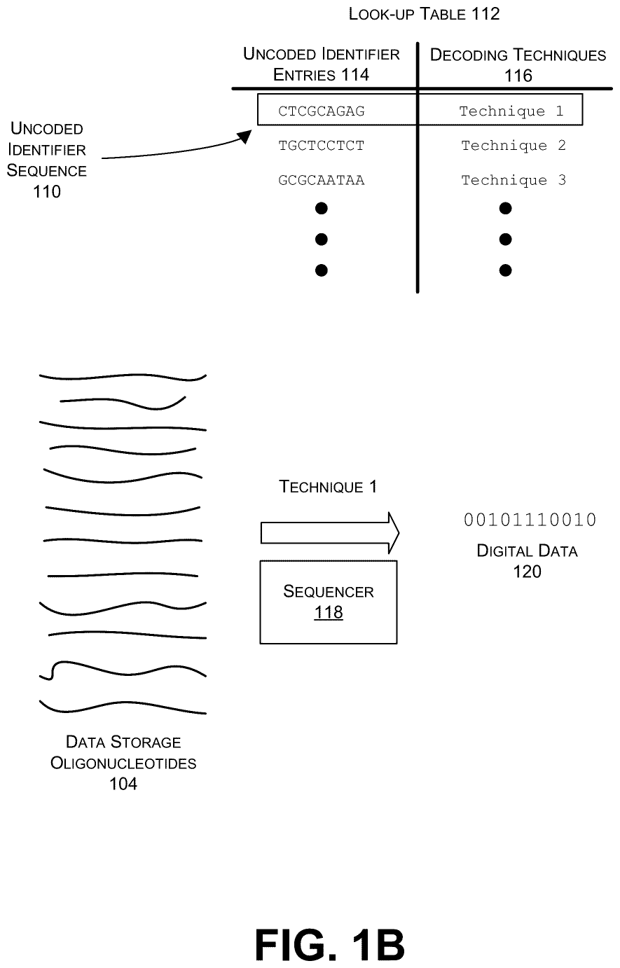

FIG. 1A shows a DNA data storage archive 100 with a label 102 on a container that holds the archive 100. The DNA data storage archive 100 is a single pool of DNA and/or other oligonucleotides that encode digital data. The archive 100 may be in any format suitable for storing oligonucleotides such as liquid (e.g., a buffered solution), dried in a pellet or onto paper, or encapsulated in silica or otherwise protected. Thus, the container may take many forms such as a tube, vial, or piece of paper. The label 102 is attached or affixed to the container and can be read by a human or machine (e.g., a barcode or QR code) without accessing the DNA in the archive 100. For example, the label 102 may be engraved on a wall of the container, written by hand, applied as a sticker, or the like. The label 102 provides the information necessary to decode the DNA data storage archive 100 or at least provides a pointer to such information. A pointer is any unambiguous reference to information (e.g., a decoding technique) available at another location. For example, a citation to a journal article and a uniform resource indicator (URI) are both pointers.

If the label 102 simply provides a unique identifier for the archive 100, then that identifier may be used to determine how to decode the DNA in the archive 100. A machine-readable code (e.g., a QR code) may provide access to an electronic resource (e.g., a webpage) that provides detailed instructions and procedures for decoding the archive 100. The label 102 may also provide metadata about the archive 100. Metadata is information other than a decoding technique that describes the contents of the archive 100. For example, metadata may include the date the archive 100 was created, the number of oligonucleotides included in the archive 100, and the like.

Potential problems arise if there is a loss of the label 102. The DNA data storage archive 100 without the label 102 still contains the same DNA and encodes the same digital data. However, even if someone knows that the container holds a DNA data storage archive 100, that person may not know which of multiple possible decoding techniques to use to recover the digital data.

The DNA data storage archive 100 contains data storage oligonucleotides 104. Even though the term “DNA” is used, it is to be understood that the archive 100 may contain oligonucleotides other than standard DNA. Oligonucleotides, and the contents of an archive 100, as used herein include DNA, ribonucleic acid (RNA), hybrids and combinations of DNA and RNA, oligonucleotides that use non-canonical and artificial bases, modified backbone structures, including fewer than all the canonical bases (e.g., only three) or more than four bases due to inclusion of artificial bases. The data storage oligonucleotides 104 are synthetic oligonucleotides created to have a specific sequence. There are multiple techniques for synthesizing data storage oligonucleotides that are known to persons of ordinary skill in the art.

Each of the data storage oligonucleotides 104 includes a data payload region 106. The data payload region 106 contains the nucleotides that encode digital data in their base sequence. Multiple techniques are known to persons of ordinary skill in the art for encoding digital data in a sequence of nucleotides. The data storage oligonucleotides 104 may also include other regions such as primer binding sites (not shown) to which PCR primers hybridize. A DNA data storage archive 100 may contain many 100s of thousands, millions, or more individual data storage oligonucleotides 104.

The data storage oligonucleotides 104 may be any length that can encode digital data and be amplified by PCR or similar techniques. For example, the data storage oligonucleotides may be between about 100-200 nucleotides long such as about 150 nucleotides long. Included in this total length are a forward primer binding site and a reverse primer binding site that may each be about 15-25 nucleotides long. The remainder of the length may be the data payload region 106. In one implementation, each data storage oligonucleotide 104 has a 20-nucleotide forward primer binding site, a 110-nucleotide long data payload region 106, and a 20-nucleotide reverse primer binding site. However, other lengths may also be used.

In order to provide a way of determining the appropriate decoding technique for the data storage oligonucleotides 104 in the absence of a usable label 102, the archive 100 also includes labeling oligonucleotides 108. The labeling oligonucleotides 108 are used to access information that may be the same as that provided by the label 102. Specifically, the labeling oligonucleotides are used to identify a decoding technique for the data storage oligonucleotides 104. The labeling oligonucleotides 108 may also provide metadata for the archive 100. The labeling oligonucleotides 108 are also synthetic oligonucleotides. The labeling oligonucleotides 108 include an uncoded identifier sequence 110. The uncoded identifier sequence 110 is a series of nucleotides that by itself, without any decoding or conversion, provides information that is used to look up the decoding technique for the data storage oligonucleotides 104. Thus, the uncoded identifier sequence 110 is only a sequence of bases, i.e., the physical arrangement of the molecules in the labeling oligonucleotides 108, and does not use any kind of translation mechanism or further processing. Each labeling oligonucleotide 108 in the same archive 108 is created to have the same uncoded identifier sequence 110.

The labeling oligonucleotides 108 are mixed in with the data storage oligonucleotides 104. Generally, there are many fewer labeling oligonucleotides 108 than data storage oligonucleotides 104. For example, there may be only one-tenth, one one-hundredth, one-one thousandth, or fewer labeling oligonucleotides 108.

In order to differentiate the labeling oligonucleotides 108 from the data storage oligonucleotides 104 the labeling oligonucleotides 108 have unique primer binding sites that are different from the primer binding sites of the data storage oligonucleotides 104. Thus, at the time of designing and synthesizing the oligonucleotides for the archive 100, all of the data storage oligonucleotides 104 are designed so that they will be amplified by different primers and will not amplify with the primer pair used for the labeling oligonucleotides 108. In some implementations, the data storage oligonucleotides 104 are designed so that no part of their sequences will hybridize to primers used for the labeling oligonucleotides 108. However, in other implementations it may be permissible for data payload region 106 of one or more of the data storage oligonucleotides 104 to hybridize to one of the primers used for amplifying the labeling oligonucleotides 108. Although this may cause off-target amplification when attempting to retrieve and sequence the labeling oligonucleotides 108, the difference in lengths of the amplification products can be used to discriminate between correct amplification of labeling oligonucleotides 108 amplification of portions of a data storage oligonucleotide 104.

The labeling oligonucleotides 108 may be any length that can contain an uncoded identifier sequence 110 and primer binding sites. Thus, the labeling oligonucleotides 108 can be PCR amplified by the use of a predetermined primer pair. This predetermined primer pair will be the same for all labeling oligonucleotides 108 in all DNA data storage archives 100. By having a standardized set of primers that are known in advance and used by all archives 100, the sequences of labeling oligonucleotides 108 can be determined even if there is no label 102.

For example, the labeling oligonucleotides 108 may be between about 100-200 nucleotides long such as about 150 nucleotides long. Included in this total length are a forward primer binding site and a reverse primer binding site that may each be about 15-25 nucleotides long. By way of example, the forward primer may be TGCCTAGCGCCTAATATGGT (SEQ ID NO: 1) and the reverse primer may be ATGTATGCGGTCAGGAGGAA (SEQ ID NO: 2). The remainder of the length may be the uncoded identifier sequence 110 which can be flanked by the primer binding sites. An example structure of a labeling oligonucleotide 108 is as follows:

[TGCCTAGCGCCTAATATGGT][uncoded identifier]
[ATGTATGCGGTCAGGAGGAA]

The labeling oligonucleotides 108 may include other regions besides the primer binding sites and the uncoded identifier sequence 110. In one implementation, each labeling oligonucleotide 108 is 150 nucleotides long and contains a 20-nucleotide forward primer binding site followed by a 110-nucleotide long uncoded identifier sequence 110 and then a 20-nucleotide reverse primer binding site. However, other lengths may also be used.

FIG. 1B shows a look-up table 112 that is used to identify a decoding technique for a given uncoded identifier sequence 110. The look-up table 112 is a record external to the DNA data storage archive 100. The look-up table 112 may be implemented as an electronic record such as a network-accessible database but could be implemented in paper form such as a book. The look-up table 112 includes at least one entry and may have many tens, hundreds, thousands, or millions of entries. Each entry in the look-up table 112 includes an uncoded identifier entry 114, which is a string of nucleotides, and a decoding technique 116. The decoding technique 116 may be identified in the look-up table 112 simply by a name, citation to published work that details the technique, or a detailed description of the decoding technique.

Reading and decoding the data stored in data-encoding oligonucleotides 104 may require performing a long sequence of steps including operations carried out in a wet lab followed by operations carried out in software. The overall steps may include PCR amplification, sequencing, and decoding. The decoding step may be necessary because the data stored in the data payload regions 106 is typically stored in encoded form to provide resilience against errors. Decoding the data stored in the archive 100 typically requires the application of one or more decoding algorithms. Various decoding algorithms such as a fountain code are known to persons of ordinary skill in the art. Some illustrative decoding techniques are described in Bornholt et al., A DNA-Based Archival Storage System, APLOS '16, 637-649 (2016) and Ping et al., Towards practical and robust DNA-based data archiving using the yin-yang codec system, Nature Computational Science 2, 234-242 (2022). The decoding technique 116 may be only the decoding algorithm for converting a string of letters representing nucleotides to a string of binary digits. Alternatively, the decoding technique 116 may include all the information identifying the decoding algorithms and decoding parameters required to decode the data stored in the archive 100. In some implementations, the look-up table 112 may include only a pointer (e.g., a unique name of a decoding technique 116) to such detailed information.

The uncoded identifier sequence 110 is a sequence of nucleotides that is determined from reading the sequences of the labeling oligonucleotides 108. Because all the labeling oligonucleotides 108 are designed to contain the same uncoded identifier sequence 110, the sequence of any single labeling oligonucleotide 108 may be sufficient. However, because there are errors in both the synthesis and sequencing of oligonucleotides, a consensus sequence derived from the sequences of multiple individual labeling oligonucleotides 108 is more likely to be accurate than the sequence of any individual oligonucleotide. Techniques for determining a consensus sequence from a plurality of sequences are known to those of ordinary skill in the art.

Even if a consensus sequence is used, there may still be errors in the nucleotide sequence of the uncoded identifiers sequence 110. Thus, the uncoded identifier sequence 110 may not match any of the uncoded identifier entries 114 listed in the look-up table 112. If this is the case, an approximate matching technique can be used to identify which uncoded identifier entry 114 is most similar to the uncoded identifier sequence 110 determined by sequencing the labeling oligonucleotides 108. For example, the uncoded identifier entry 114 with the smallest edit distance (e.g., the fewest number of insertions, deletions, and substitutions) from the uncoded identifier sequence 110 may be used as the match.

Once a decoding technique is identified from the look-up table 112, that decoding technique 116 can then be used to decode the data storage oligonucleotides 104 and recover digital data 120. Recovering digital data from the data payload regions 106 may include amplification of some or all of the data storage oligonucleotides by PCR. The primers to be used for this amplification may be identified by the decoding technique 116. Once amplified, if necessary, the data storage oligonucleotides 104 are sequenced by a sequencer 118. The sequencer 118 may use any type of known or later-developed technique for sequencing oligonucleotides such as sequencing-by-synthesis or nanopore sequencing. The nucleotide sequences generated by the sequencer 118 are then converted into digital data 120.

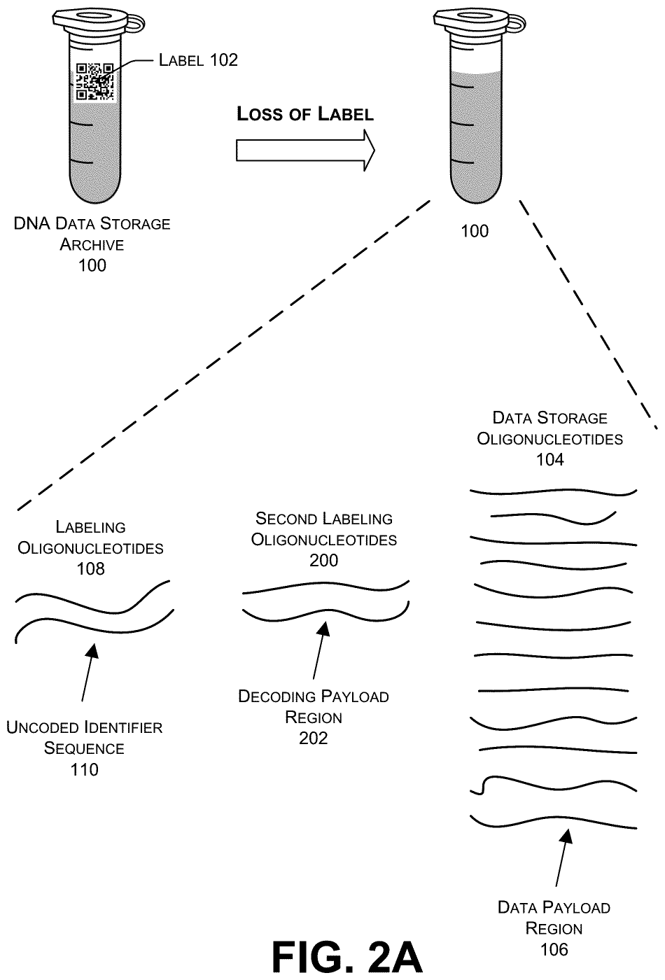

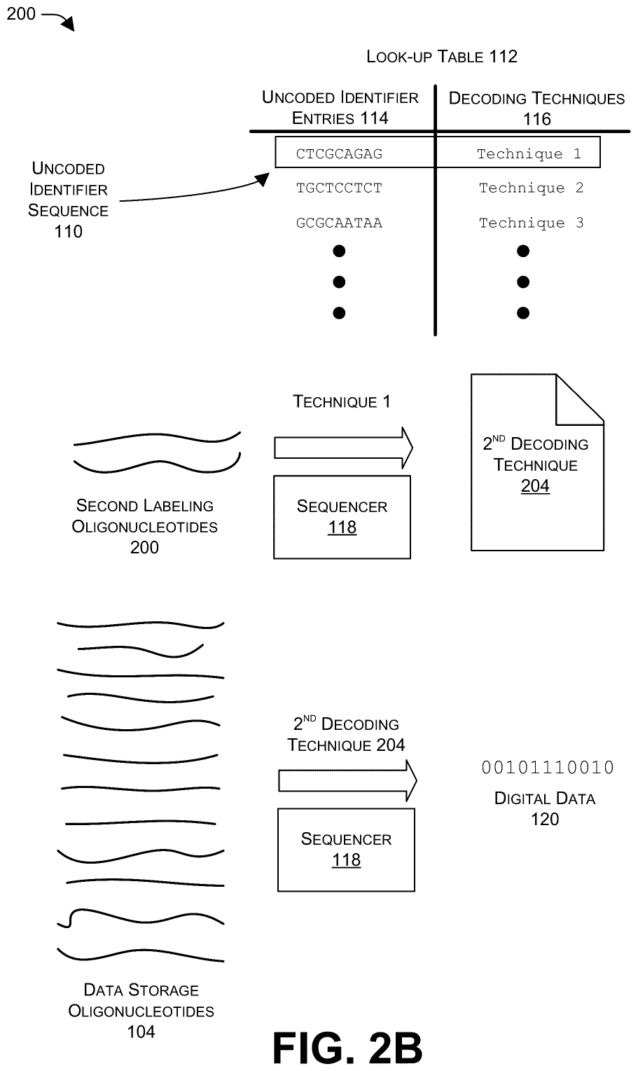

FIG. 2A shows a DNA data storage archive 100 and the oligonucleotides contained within. This is similar to FIG. 1A but FIG. 2A differs by the inclusion of second labeling oligonucleotides 200. The second labeling oligonucleotides 200 are a second set of labeling oligonucleotides in addition to the labeling oligonucleotides 108 introduced in FIG. 1A. The second labeling oligonucleotides 200 are also synthetic oligonucleotides. In the implementation shown in FIGS. 2A and 2B, the labeling oligonucleotides 108 are used to decode the second labeling oligonucleotides 200 which in turn are used to decode the data storage oligonucleotides 104. Adding second labeling oligonucleotides 200 provides greater flexibility and customizability compared to using only one type of labeling oligonucleotide.

Thus, in this implementation, the DNA data storage archive will contain labeling oligonucleotides 108, second labeling oligonucleotides 200, and data storage oligonucleotides 104. Typically, there will be many more data storage oligonucleotides than labeling oligonucleotides 108 and second labeling oligonucleotides. There may be approximately the same or there may be different amounts of labeling oligonucleotides 108 and second labeling oligonucleotides 200. There may be, for example, about ten times, 100 times, 1000 times, or more data storage oligonucleotides 104 than second labeling oligonucleotides 200.

The second labeling oligonucleotides 200 each contain a decoding payload region 202. The decoding payload region 202 contains nucleotides in a payload region that, when decoded, provides a decoding technique for the data storage oligonucleotides 104. The decoding payload region 202 may encode the same information as the label 102. If this information cannot be encoded in the length of a single decoding payload region 202, then the encoded information may be partitioned into multiple payload regions. However, each of the second labeling oligonucleotides 200 will have the same primer binding sites.

The second labeling oligonucleotides 200 may be any length that can contain a decoding payload region 202 and primer binding sites. For example, the labeling oligonucleotides 108 may be between about 100-200 nucleotides long such as about 150 nucleotides long. Included in this total length are a forward primer binding site and a reverse primer binding site that may each be about 15-25 nucleotides long. The forward and reverse primer binding sites in the second labeling oligonucleotides 200 are different than the forward and reverse primer binding sites in the labeling oligonucleotides 108. Thus, primers that hybridize to the primer binding sites of either the labeling oligonucleotides 108 or the second labeling oligonucleotides 200 will not hybridize to the other. However, it is possible that a primer for the labeling oligonucleotides 108 may hybridize to the decoding payload region 202 on one of the second labeling oligonucleotides 200.

Additionally, the data storage oligonucleotides 104 are designed so that the primer binding sites do not hybridize to the primers used to amplify either the labeling oligonucleotides 108 or the second labeling oligonucleotides 200. In some implementations, the data storage oligonucleotides 104 are designed so that they do not include any sequences that hybridize to these primers. However, it may be acceptable in some implementations for the primers that amplify either the labeling oligonucleotides 108 or the second labeling oligonucleotides 200 to hybridize with the data payload region 106 of some of the data storage oligonucleotides 104. Thus, the DNA data storage archive may be designed with two pairs of reserved primers one for the labeling oligonucleotides 108 and one for the second labeling oligonucleotides 200.

By way of example, the forward primer may be TGACCGCTACGATTAGACCA (SEQ ID NO: 3) and the reverse primer may be GCAAAGCGGTTGTCTTCTCT (SEQ ID NO: 4). The remainder of the length may be the decoding payload region 202 which can be flanked by the primer binding sites. An example structure of a second labeling oligonucleotide 200 is as follows:

[TGACCGCTACGATTAGACCA][decoding payload region]
[GCAAAGCGGTTGTCTTCTCT]

The second labeling oligonucleotides 200 may include other regions besides the primer binding sites and the decoding payload region 202. In one implementation, each second labeling oligonucleotide 200 is 150 nucleotides long and contains a 20-nucleotide forward primer binding site followed by a 110-nucleotide long decoding payload region 202 and then a 20-nucleotide reverse primer binding site. However, other lengths may also be used.

The primer used to amplify the second labeling oligonucleotides 200 may also be predetermined primers with sequences that are known and published as part of a DNA data storage archive bootstrapping protocol. However, in other implementations, the sequences of the primers for the second labeling oligonucleotides 200 may not be known in advance and instead are found from the uncoded identifier sequence 110 in the labeling oligonucleotides 108. The labeling oligonucleotides 108 may also be referred to as “sector 0” oligonucleotides. The second labeling oligonucleotides 200 may also be referred to as “sector 1” oligonucleotides.

FIG. 2B shows how the labeling oligonucleotides 108 and the second labeling oligonucleotides 200 are used to decode the data storage oligonucleotides 104. An uncoded identifier sequence 110 is obtained and used to query a look-up table 112 as described above. This identifies a decoding technique 116. That decoding technique is used to decode the decoding payload region 202 of the second labeling oligonucleotides 200. Thus, unlike the technique illustrated in FIG. 1, the decoding technique 116 identified from the look-up table 112 is used not to decode the data storage oligonucleotides 104, but to decode the second labeling oligonucleotides 200.

Decoding may include amplifying the second labeling oligonucleotides 200 with primers specific to those oligonucleotides and sequencing the amplification product with a sequencer 118. Because the nucleotides in the decoding payload region 202 are not uncoded identifiers they can and are used to encode arbitrary information. The results of decoding the decoding payload region 202 is human- or machine-readable information that provides a second decoding technique 204. Thus, the nucleotides in the second labeling oligonucleotides 200 may decode into words in a natural language (e.g., English) that describe the second decoding technique 204. Alternatively, the nucleotides of the second labeling oligonucleotides 200 may decode into a series of binary digits that can be interpreted by a computer as the second decoding technique 204.

The decoding technique 116 identified from the look-up table 112 is limited to only those decoding techniques 116 that have been entered into the look-up table 112. But the second decoding technique 204 may be any decoding technique and can be freely specified with as much detail and additional described information as the creator of the second labeling oligonucleotides 200 desires. This provides additional flexibility and customizability beyond the decoding techniques 116 recorded in the look-up table 112. The decoding technique 116 obtained from the look-up table 112 and the second decoding technique 204 will typically be different decoding techniques but this is not necessarily the case. The second decoding technique 204 is then used to decode the data storage oligonucleotides 104 and recover the digital data 120 as described above.

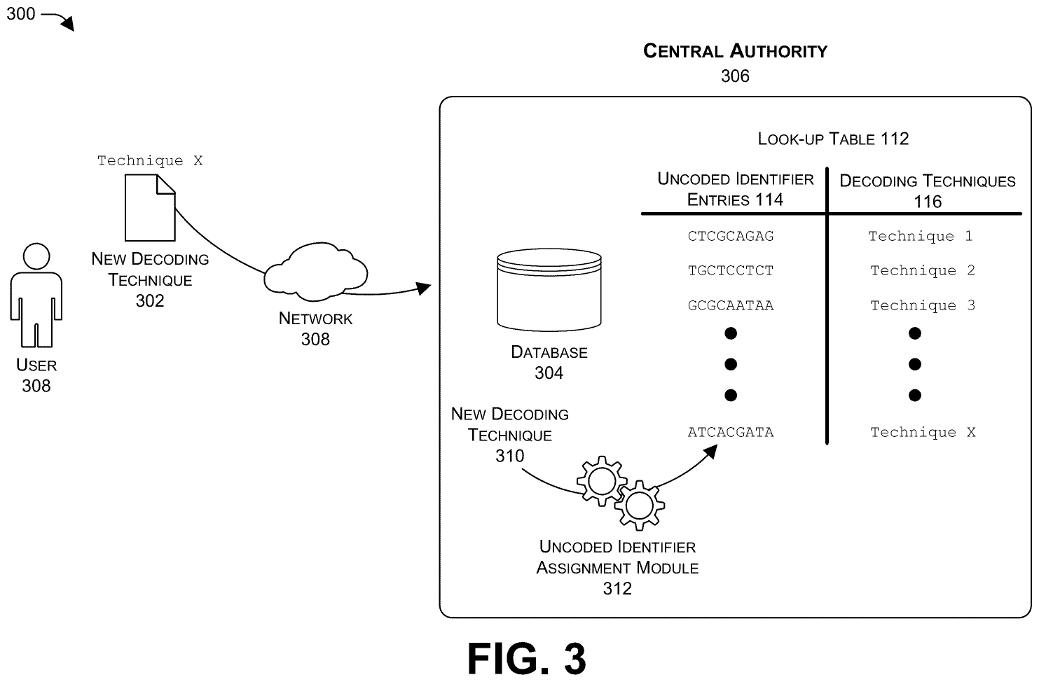

FIG. 3 is a diagram 300 that shows the addition of a new decoding technique 302 to a look-up table 112. The look-up table 112 may be the same as the look-up table 112 shown in FIGS. 1 and 2. In an implementation, the look-up table 112 is maintained in a database 304 that is hosted by a central authority 306 and accessible via a network 308. The database 304 and the look-up table 112 may be implemented as any type of hardware and data structures that are capable of uniquely associating an uncoded identifier entry 114 with a decoding technique 116.

The central authority 306 is an organization or entity that maintains the look-up table 112 and makes it available to others. For example, the database 304, and thus the look-up table 112, may be available through an application programming interface (API) call. The central authority 306 can also provide a public specification or standard for DNA data storage archives. This standard provides a set of rules for generating DNA data storage archives that can be accessed using the bootstrapping techniques provided in this disclosure. Thus, the central authority 306 may play a role for DNA data storage archives that is similar to how the Internet Corporation for Assigned Names and Numbers (ICANN) manages the Internet Protocol address spaces.

The network 308 may be any type of communications network such as the Internet. Thus, the database 304 that contains the look-up table 112 may be widely accessed. Although shown as a single database 304 in the diagram 300, the contents of the database 304 may be distributed across multiple different pieces of hardware at different physical locations—a “cloud” implementation. There may also be multiple redundant copies of the database 304.

A user 308 may provide the new decoding technique 302 to the central authority 306 via the network 308. The description of the new decoding technique 302 may include any of the information described above for decoding techniques including any information that would be found in a conventional label of a DNA data storage archive. However, this communications path is not required and the user 308 may provide the new decoding technique 302 in any way that information is communicated such as, for example, mailing a description printed on paper. The new decoding technique 302 is not necessarily a newly developed or novel decoding technique (although it may be). It is simply a decoding technique that is not yet included in the look-up table 112.

An uncoded identifier assignment module 312 assigns a series of nucleotides to the new decoding technique 302 that will function as the uncoded identifier entry 114. The uncoded identifier assignment module 312 may be implemented in software, firmware, hardware, or a combination. The look-up table 112 may contain many thousands or millions of decoding techniques 116. Each is assigned a different series of nucleotides as its uncoded identifier entry 114. In some implementations, an uncoded identifier entry may contain between about 60-160 nucleotides such as about 110 nucleotides. This provides many possible values (e.g., 4¹¹⁰≈1.7×10⁶⁶) for uncoded identifier entries. However, to avoid ambiguity when querying the look-up table 112, it is preferable to make each uncoded identifier entry 114 significantly different from all others.

Thus, the uncoded identifier assignment module 312 may consider all uncoded identifier entries 114 already in the look-up table 112 and identify a new series of nucleotides that has at least a threshold difference from the existing entries. This difference may be measured by edit distance. For example, a threshold value of the edit distance could be at least twice the expected number of errors introduced by the sequencing technology used to read the labeling oligonucleotides. The error rate is the percentage of base calls that on average will be incorrect in a consensus sequence generated from combining the reads of multiple oligonucleotides with the same sequence. The maximum number of errors expected is the error rate multiplied by the length of the oligonucleotide. For an oligonucleotide with 100 bases, an error rate of 15% would lead to a maximum of 15 errors expected. Twice the maximum number of errors in this example is 30. Thus, the threshold edit distance would be 30. In one implementation, the threshold edit distance is twice the maximum number of errors plus one. So it would be 31. Adding one helps avoid collisions during approximate matching in the event that the number of errors in a sequence exactly equals the maximum expected number.

In another implementation, the uncoded identifier module 312 may determine a series of nucleotides to use as the next uncoded identifier entry 114 that has the maximal edit distance from all entries already in the look-up table 112 rather than just a threshold difference. The maximal edit distance is a nucleotide sequence that has as large as possible (accounting for other considerations and limitations) an edit distance from all other entries in the look-up table 112.

The uncoded identifier assignment module 312 can also select sequences of nucleotides for uncoded identifiers that have certain characteristics which make them more suitable for use in a DNA data storage archive. For example, nucleotide sequences that hybridize to primer binding sites of labeling oligonucleotides, second labeling oligonucleotides, and data storage oligonucleotides may be excluded. Nucleotides sequences with homopolymers or homopolymer runs (e.g., more than two or three) may be excluded. Allowable sequences for uncoded identifiers may also be limited to sequences with specific GC content. For example, allowable sequences may be limited to only sequences with about 50% GC content such as between 45-55% GC content. Additional considerations that may be accounted for by the uncoded identifier assignment module 312 include selecting nucleotide sequences that do not form secondary structures such as hairpins.

Once an uncoded identifier entry 114 is selected for the new decoding technique 302 both are added to the look-up table 112. The user 308 may receive a notification that the new decoding technique 302 was added. The notification may include the uncoded identifier entry 114 associated with the new decoding technique 302.

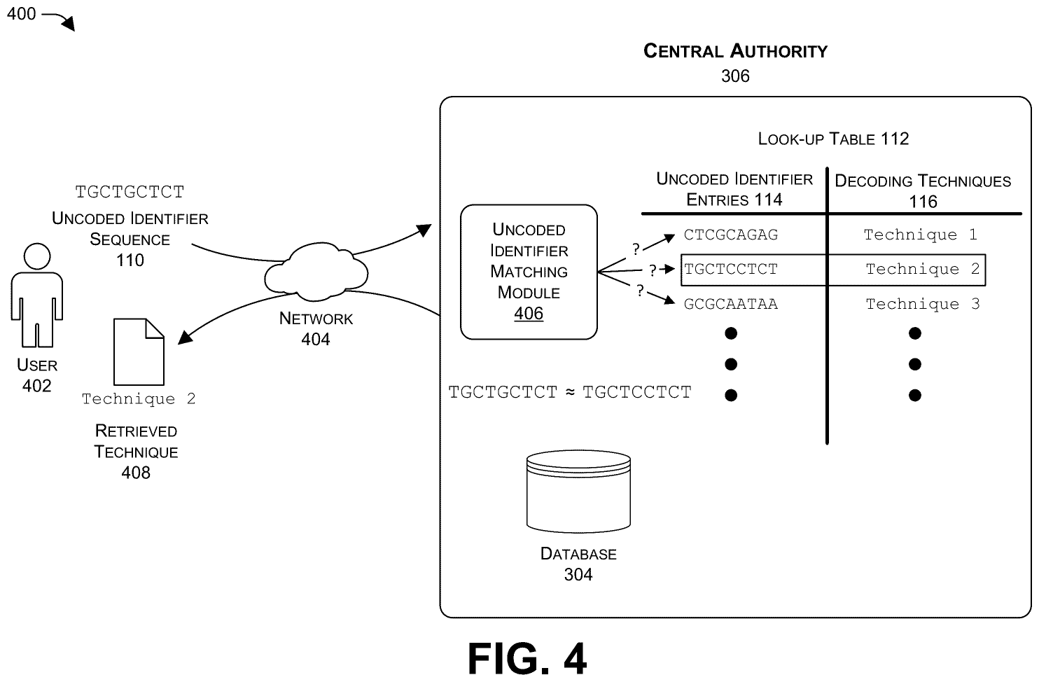

FIG. 4 is a diagram 400 that shows a user 402 querying the central authority 306 with an uncoded identifier sequence 110. As described above, the uncoded identifier sequence 110 is obtained from sequencing the labeling oligonucleotides. Although all of the labeling oligonucleotides in a given archive are designed to contain the same uncoded identifier sequence 110, there may be errors in one or both of the synthesis and sequencing of the labeling oligonucleotides. The uncoded identifier sequence 110 may be the sequence of a single molecule, but more commonly it will be a consensus sequence derived from the sequences of many labeling oligonucleotides. Creating a consensus sequence reduces many of the errors that can exist in individual sequences but may not be entirely error free. Thus, the uncoded identifier sequence 110 may not be an exact match for any of the uncoded identifier entries 114 in the look-up table 112. The uncoded identifier sequence 110 may be a noisy sequence that contains insertion, deletion, and substitution errors.

The central authority 306, database 304, and look-up table 112 may be implemented the same as in FIG. 3. If the database 304 is network accessible, the user 402 can provide the uncoded identifier sequence 110 via a network 404. The network 404 may be any type of communications network such as the Internet. It is also possible for the user 402 to access a locally cached copy of the database 304 and obtain a decoding technique without communication with the central authority 306 across a network 404.

An uncoded identifier matching module 406 receives the uncoded identifier sequence 110 and attempts to match it with one of the entries in the look-up table 112. The uncoded identifier matching module 406 may be a component of the database 304. The uncoded identifier matching module 406 may be implemented in software, firmware, hardware, or a combination. The uncoded identifier matching module 406 may compare the uncoded identifier sequence 110 to each of the uncoded identifier entries 114 in turn. Exact matches can be readily identified by comparison of the two nucleotide sequence strings. In one implementation, the look-up table 112 is queried for exact matches first and approximate matches are considered only if there is no exact match.

Approximate matches are more difficult to identify. The goal is to determine which entry in the look-up table 112 is sufficiently similar to the uncoded identifier sequence 110 to be considered a match. One technique is to calculate the edit distance from the uncoded identifier sequence 110 to each uncoded identifier entry 114. The uncoded identifier entry 114 with the smallest edit distance from the uncoded identifier sequence 110 is selected as the match. The associated decoding technique 116 is then identified from the look-up table 112.

However, there may be a maximum allowable edit distance (e.g., 5, 10, 15) for identifying a match. If an uncoded identifier sequence 110 is not within that maximum allowable edit distance of any of the uncoded identifier entries 114 then there is no match.

It may also not be necessary to calculate the edit distance from every uncoded identifier entry 114 in the look-up table 112. Because the uncoded identifier entries 114 may be designed so that each has at least a minimum level of dissimilarity from each other, an edit distance less than this minimum level of dissimilarity may be interpreted as a match. Once a match is identified, the comparison may stop without calculating an edit distance from the remaining uncoded identifier entries 114.

Once one of the decoding techniques 116 is identified from the look-up table 112, it is returned to the user 402 via the network 404. This is referred to as the retrieved technique 408. The user 402 can then use the retrieved technique 408 to recover digital data from the data storage oligonucleotides as shown in FIG. 1B or to decode the payloads of second labeling oligonucleotides as shown in FIG. 2B.

The retrieved technique 408 may be provided to the user 402 in any number of different ways such as displaying instructions for performing the technique on a webpage. In some implementations, all or part of the retrieved technique 408 may be provided as machine-readable code that can be executed by a device. For example, the retrieved technique 408 may include instructions that cause a microfluidics system, laboratory robotics system, or the like to perform physical operations to decode oligonucleotides such as PCR and sequencing. The retrieved technique 408 may additionally or alternatively provide instructions that software can use to decode a nucleotide sequence provided by a sequencer into a series of binary digits.

### Illustrative Processes

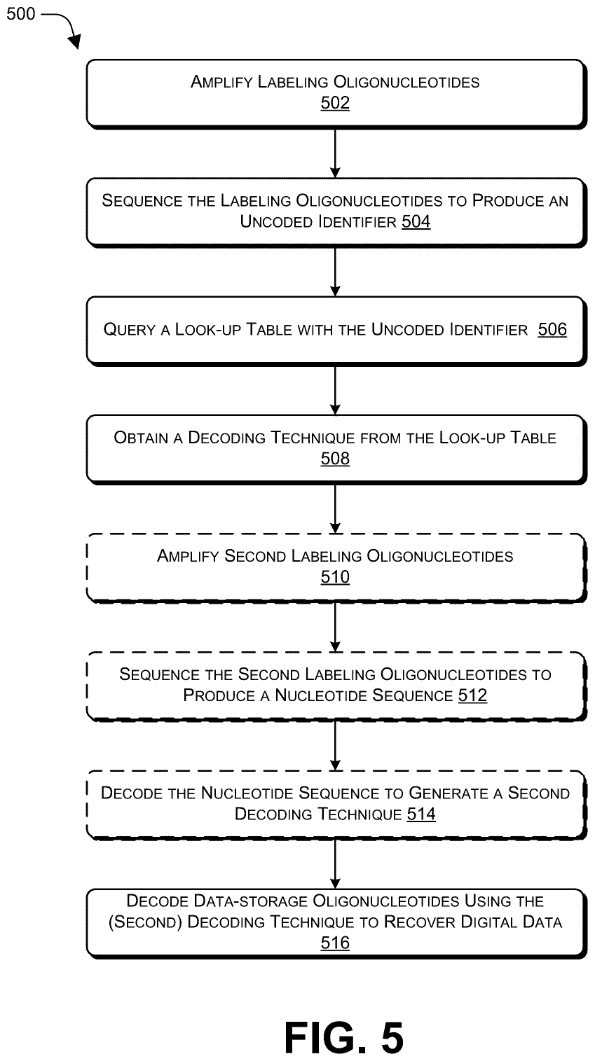

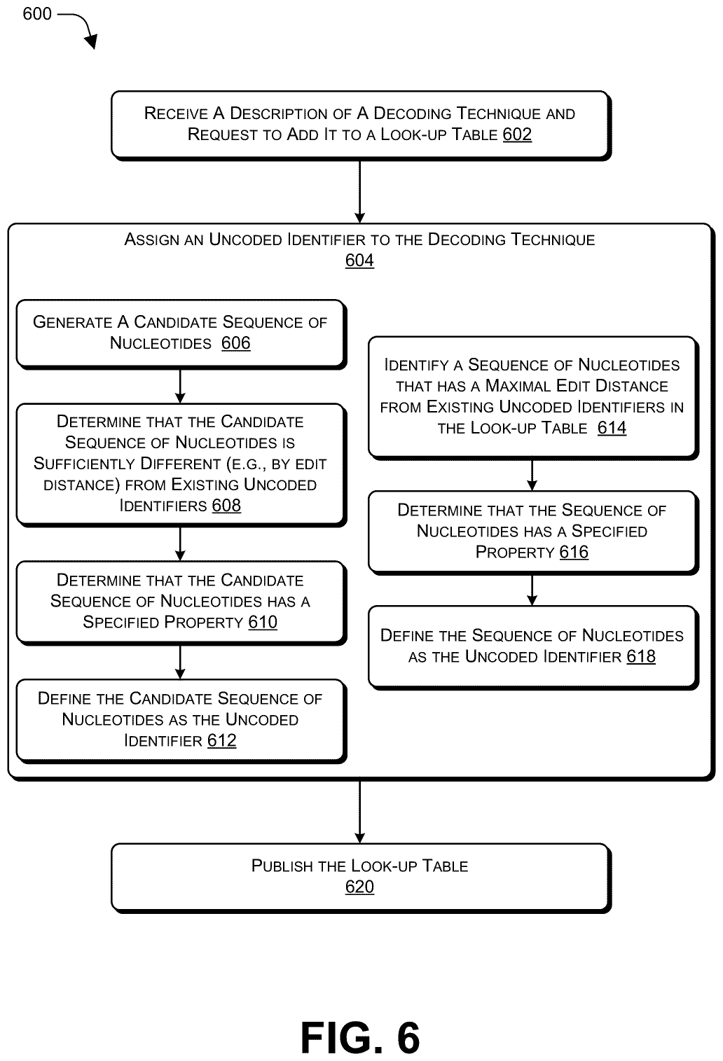

For ease of understanding, the processes discussed in FIGS. 5 and 6 are delineated as separate operations represented as independent blocks. However, these separately delineated operations should not be construed as necessarily order dependent in their performance. The order in which a process is described is not intended to be construed as a limitation, and any number of the described process blocks may be combined in any order to implement the process or an alternate process. Moreover, it is also possible that one or more of the provided operations is modified or omitted.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, special-purpose digital logic, and any combination thereof. It should be appreciated that more or fewer operations can be performed than shown in the figures and described herein. These operations can also be performed in a different order than those described herein.

FIG. 5 shows a process 500 for identifying a decoding technique (“bootstrapping”) for synthetic data storage oligonucleotides that encode digital data. Process 500 may be implemented with any of the systems and techniques shown in FIGS. 1 and 2.

At operation 502, labeling oligonucleotides in a DNA data storage are amplified through PCR using a predetermined primer pair to generate a labeling oligonucleotide amplification product.

At operation 504, the labeling oligonucleotide amplification product is sequenced to produce an uncoded identifier sequence. The uncoded identifier sequence is a sequence of nucleotides in the labeling oligonucleotides. Specifically, the uncoded identifier sequence may come from a payload region of the labeling oligonucleotides. The payload region is between two primer binding sites that hybridize to the predetermined primer pair. In some implementations, the uncoded identifier sequence is a consensus sequence derived from the sequences of multiple different individual labeling oligonucleotides. This consensus sequence may be a noisy sequence that includes insertion, deletion, and/or substitution errors. Thus, the consensus sequence may not exactly match the intended payload of the labeling oligonucleotides.

At operation 506, a look-up table is queried with the uncoded identifier sequence. The look-up table may be maintained in an electronic database that is accessible over a network such as the Internet. The look-up table may contain a plurality of different decoding techniques each uniquely associated with a different uncoded identifier entry. For example, an uncoded identifier matching module 406 as shown in FIG. 4 may be used to query the look-up table 112. In one implementation, the look-up table is queried by calculating an edit distance between the uncoded identifier sequence and at least one uncoded identifier entry in the look-up table. Edit distance as used herein may refer to Levenshtein edit distance. In one implementation, an edit distance is computed for each uncoded identifier entry and the match is the entry with the smallest edit distance. In another implementation, the edit distance is calculated for entries in the look-up table until a match is found without calculating an edit distance for every entry.

At operation 508, a decoding technique is obtained from the look-up table. In the look-up table, the sequence of nucleotides that is the uncoded identifier is uniquely associated with the decoding technique.

If only a single set of labeling oligonucleotides is included in the DNA data storage archive as shown in FIG. 1, process 500 will move next to operation 516. Operations 510, 512, and 514 are included in process 500 only if there are second labeling oligonucleotides as shown in FIG. 2.

At operation 510, if present in the DNA data storage archive, second labeling oligonucleotides are amplified through PCR using a second predetermined primer pair. This second primer pair is different than the primer pair used to amplify the labeling oligonucleotides at operation 502. In an implementation, the second predetermined primer pair is specified in the decoding technique. Thus, the primers for amplifying the second labeling oligonucleotides may not be known until the decoding technique is obtained.

At operation 512, the second labeling oligonucleotides are sequenced to produce one or more nucleotide sequences. The nucleotide sequence may be a consensus sequence derived from the sequences of multiple individual second labeling oligonucleotides. This nucleotide sequences encode information but the second labeling oligonucleotides are distinct from the data storage oligonucleotides. The amount of information encoded may be larger than that which can be stored in a single oligonucleotide. Accordingly, it may be split across multiple second labeling oligonucleotides that each encode a portion of the total information. This information may be encoded with an error-correcting code to assist with resilience against synthesis and sequencing errors. Consequently, reading the information encoded in the second labeling oligonucleotides requires knowledge of the decoding algorithms and parameters to convert the nucleotide sequence into usable information.

At operation 514, the nucleotide sequence is decoded with the decoding technique obtained at operation 508 to generate a second decoding technique. Decoding the nucleotide sequence may provide human- or machine-readable information that contains the second decoding technique. This second decoding technique is not obtained from the look-up table. It may be described by the decoded content of the nucleotide sequence. However, in an implementation, a different look-up table or similar index may be used to obtain the details of the second decoding technique.

At operation 516, data storage oligonucleotides are decoded using the decoding technique from operation 508, or if second labeling oligonucleotides are present, decoded using the second decoding technique from operation 514. The decoding recovers digital data from the data storage oligonucleotides.

FIG. 6 shows a process 600 for adding a decoding technique to a look-up table. Process 600 may be implemented with any of the systems and techniques shown in FIG. 3.

At operation 602, a description of a decoding technique and a request to add the decoding technique to a look-up table is received. This description and request may come from a user who wishes to add the decoding technique to a publicly available repository of decoding techniques. The decoding technique may include all the same information that is present on a label for a DNA data storage archive. The decoding technique may describe at least techniques for converting nucleotide sequences to digital data while accounting for insertion, deletion, and substitution errors in the nucleotide sequences. The decoding techniques may also describe a primer pair used for PCR amplification of data storage oligonucleotides comprising data payload regions that encode digital data. Thus, the decoding technique may be used to decode data storage oligonucleotides.

At operation 604, an uncoded identifier entry is assigned to the decoding technique. The uncoded identifier is a sequence of nucleotides. The sequence of nucleotides may be any length that can be used to uniquely identify one encoding technique out of many in the presence of errors and that can be accurately synthesized. For example, the nucleotide sequence may include between about 60 to about 160 nucleotides such as about 110 nucleotides. There are multiple possible ways that an uncoded identifier entry could be assigned to a decoding technique. One way is described in operations 606 to 612. Another way is described in operations 614 to 618. However, the possible techniques are not limited to these two.

At operation 606, a candidate sequence of nucleotides is generated. The candidate sequence may be generated by randomly generating a sequence of nucleotides of a specified length (e.g., 110).

At operation 608, it is determined that the candidate sequence of nucleotides is sufficiently different from existing uncoded identifier entries in the look-up table. The extent of the difference may be measured by calculating an edit distance between the candidate sequence of nucleotides and sequences of nucleotides that are the existing uncoded identifier entries. If it is determined that the edit distance from each existing uncoded identifier entry is greater than a threshold amount, then the candidate sequence may still be potentially used as an uncoded identifier. If, however, the edit distance is less than the threshold amount, the candidate sequence is discarded. Process 600 may restart at operation 606 by generating another candidate sequence.

The threshold amount may be any amount that allows an uncoded identifier sequence from labeling oligonucleotides to be uniquely matched with a single uncoded identifier entry. The threshold amount may be an edit distance that is at least double the maximum possible edit distance that can be introduced by the insertion, deletion, and substitution errors in the labeling oligonucleotides. For example, if errors could cause the uncoded identifier sequence to differ from the actual intended uncoded identifier by an edit distance of 15, then the threshold amount would be an edit distance of at least 30 from all other entries. In some implementations, this threshold amount may be an edit distance of 30, 31, 32, 33, 34, 35, 36, 37, 38, 39, or 40.

At operation 610, it is determined that the candidate sequence of nucleotides has a specified property. The specified property is any one or more other properties that improve the functioning of the labeling oligonucleotides. If the candidate sequence does not have all of the properties that are specified it is discarded and not used. Process 600 may restart at operation 606 by generating another candidate sequence.

The specified property may be the property of not containing a primer binding site for primers used for PCR amplification of labeling oligonucleotides. Thus, the uncoded identifier cannot contain a nucleotide sequence that would hybridize with one of the primers used to amplify the labeling oligonucleotides. Another possible specified property is not containing homopolymers or not containing a homopolymer run of more than two, three, four, or some other determined value. Homopolymers, particularly runs of three or more, may make the oligonucleotides more difficult to sequence accurately. The specified property may be GC content or about 50% such as between about 45% and 55%. Another specified property may be a sequence that does not form secondary structures such as hairpins. Persons of ordinary skill in the art will be able to readily identify and use software tools that can analyze nucleotide sequences and predict the formation of secondary structures.

At operation 612, the candidate sequence of nucleotides is defined as the uncoded identifier because it meets all of the requirements for a new uncoded identifier.

At operation 614, for an alternative technique of assigning an uncoded identifier, a sequence of nucleotides that has a maximal edit distance from existing uncoded identifier entries in the look-up table.

At operation 616, it is determined that the sequence of nucleotides has a specified property. This operation may be the same as operation 610. If it does not have any of the specific properties that are required, the sequence of nucleotides is discarded even though it has the maximal edit distance from the existing uncoded identifier entries.

At operation 618, the sequence of nucleotides is defined as the uncoded identifier. The next description of a decoding technique that is added to the look-up table can be assigned a sequence of nucleotides in the same way. Thus, each addition to the look-up table will be associated with an uncoded identifier that is as dissimilar as possible from all existing uncoded identifiers while also satisfying any other rules such as having specified properties.

At operation 620, the look-up table is published. It may be published by a central authority that maintains standards and specifications for implementation of DNA data storage archives. Publication may be making the look-up table available on a network-accessible database. The look-up table may be configured to receive queries over a network, the queries will include a nucleotide sequence obtained from sequencing labeling oligonucleotides. Publication could also be in the form of a book or printed material that is not in electronic form. The published look-up table may be updated and a new version generated each time a decoding technique is added.

### Illustrative Computer Architecture

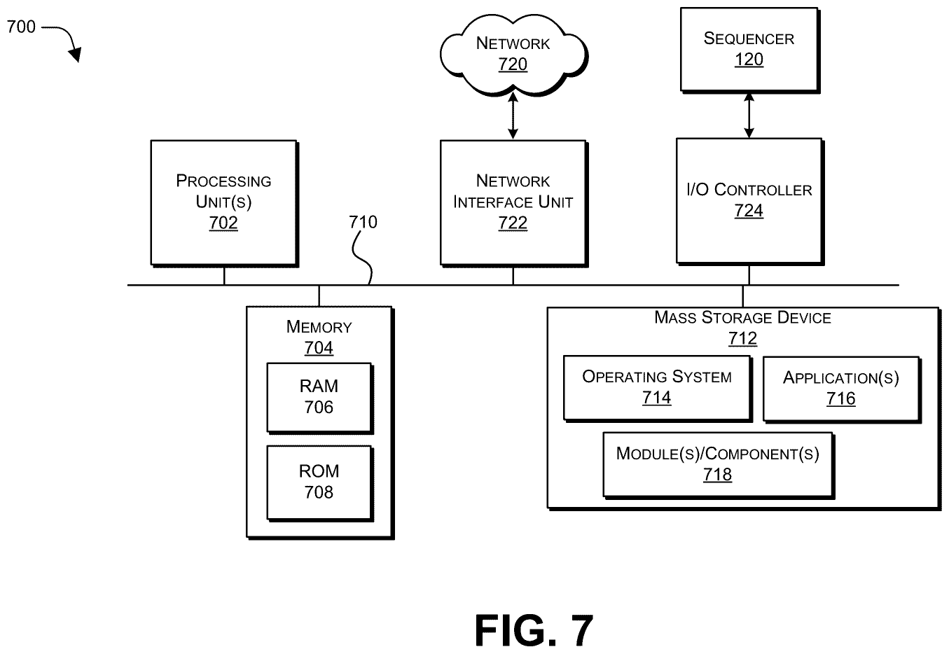

FIG. 7 shows details of an illustrative computer architecture 700 for a device, such as a computer or a server capable of executing computer instructions (e.g., a module or a component described herein). For example, computer architecture 700 may represent a computer that maintains the database 304 shown in FIGS. 3 and 4. The computer architecture 700 illustrated in FIG. 7 includes one or more processing unit(s) 702, memory 704, including a random-access memory 706 (“RAM”) and a read-only memory (“ROM”) 708, and a system bus 710 that couples the memory 704 to the processing unit(s) 702. The processing units(s) 702 may also comprise or be part of a processing system, processor, or hardware logic circuitry. In various examples, the processing unit(s) 702 of the processing system are distributed. Stated another way, one processing unit 702 of the processing system may be located in a first location (e.g., a rack within a datacenter) while another processing unit 702 of the processing system is located in a second location separate from the first location.

Processing unit(s) 702 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like.

A basic input/output system containing the basic routines that help to transfer information between elements within the computer architecture 700, such as during startup, is stored in the ROM 708. The computer architecture 700 further includes a mass storage device 712 for storing an operating system 714, application(s) 716, modules/components 718, and other data described herein. The modules/components 718 may include the uncoded identifier assignment module 312 shown in FIG. 3 and/or the uncoded identifier matching module 406 shown in FIG. 4.

The mass storage device 712 is connected to the processing unit(s) 702 through a mass storage controller connected to the bus 710. The mass storage device 712 and its associated computer-readable media provide non-volatile storage for the computer architecture 700. Although the description of computer-readable media contained herein refers to a mass storage device, it should be appreciated by those skilled in the art that computer-readable media can be any available computer-readable storage medium or communications medium that can be accessed by the computer architecture 700.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer-readable storage media are tangible and/or physical forms of media included in a device and/or hardware component that is part of a device or external to a device, including RAM, static random-access memory (SRAM), dynamic random-access memory ((DRAM), phase-change memory (PCM), ROM, erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network-attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage medium does not include communication medium. That is, computer-readable storage media does not include communications media and thus excludes media consisting solely of a modulated data signal, a carrier wave, or a propagated signal, per se.

According to various configurations, the computer architecture 700 may operate in a networked environment using logical connections to remote computers through the network 720. The network 720 may be the same as the network 308 shown in FIG. 3 or the network 404 shown in FIG. 4. The computer architecture 700 may connect to the network 720 through a network interface unit 722 connected to the bus 710. An I/O controller 724 may also be connected to the bus 710 to control communication in input and output devices.

It should be appreciated that the software components described herein may, when loaded into the processing unit(s) 702 and executed, transform the processing unit (s) 702 and the overall computer architecture 700 from a general-purpose computing system into a special-purpose computing system customized to facilitate the functionality presented herein. The processing unit(s) 702 may be constructed from any number of transistors or other discrete circuit elements, which may individually or collectively assume any number of states. More specifically, the processing unit(s) 702 may operate as a finite-state machine, in response to executable instructions contained within the software modules disclosed herein. These computer-executable instructions may transform the processing unit(s) 702 by specifying how the processing unit(s) 702 transitions between states, thereby transforming the transistors or other discrete hardware elements constituting the processing unit(s) 702.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. This clause generally describes a method of retrieving a codec from an unlabeled archive. This clause is illustrated in one example implementation by FIG. 3. A method of identifying a decoding technique for data storage oligonucleotides (104) that encode digital data (120), the method comprising: amplifying (502) labeling oligonucleotides (108) in a DNA data storage archive (100) by polymerase chain reaction (PCR) amplification using a predetermined primer pair to generate a labeling oligonucleotide amplification product; sequencing (504) the labeling oligonucleotide amplification product to produce an uncoded identifier sequence (110) which is a sequence of nucleotides in the labeling oligonucleotides; querying (506) a look-up table (112) with the uncoded identifier sequence; and obtaining (508) from the look-up table the decoding technique (116; 408) uniquely associated with the uncoded identifier.

Clause 2. This clause generally describes implementation with only sector 0 oligos. The method of clause 1, further comprising decoding the data storage oligonucleotides in the DNA data storage archive using the decoding technique, wherein the decoding recovers the digital data from the data storage oligonucleotides.

Clause 3. This clause generally describes implementation with a sector 1 oligo. The method of clause 1, further comprising: amplifying second labeling oligonucleotides in the DNA data storage archive by PCR amplification using a second predetermined primer pair to generate a second labeling oligonucleotide amplification product: sequencing the second labeling oligonucleotide amplification product to produce a nucleotide sequence; and decoding the nucleotide sequence with the decoding technique to generate human- or machine-readable information that comprises a second decoding technique.

Clause 4. The method of clause 3, wherein the second predetermined primer pair is specified in the decoding technique.

Clause 5. The method of clause 3 or 4, further comprising decoding data storage oligonucleotides in the DNA data storage archive using the second decoding technique, wherein the second decoding technique recovers the digital data from the data storage oligonucleotides.

Clause 6. The method of clause 3 to 5, wherein human- or machine-readable information decoded from the second labeling oligonucleotides comprises metadata for the DNA data storage archive.

Clause 7. The method of clause 1 to 6, wherein the look-up table comprises a plurality of different decoding techniques each uniquely associated with a different uncoded identifier entry.

Clause 8. The method of clause 7, wherein querying the look-up table comprises calculating an edit distance between the uncoded identifier sequence and at least one uncoded identifier entry in the look-up table.

Clause 9. This clause generally describes a storage archive itself with the different types of oligos. A DNA data storage archive (100) comprising: labeling oligonucleotides (108) comprising an uncoded identifier sequence (110) flanked by primer binding sites for a predetermined primer pair, wherein the uncoded identifier sequence is a sequence of nucleotides and the uncoded identifier sequence is uniquely associated in an external look-up table (112) with a decoding technique (116); and data storage oligonucleotides (104) comprising data payload regions that encode digital data (120).

Clause 10. The DNA data storage archive of clause 9, wherein the primer binding sites are not found in the data storage oligonucleotides.

Clause 11. This clause generally describes an implementation with only sector 0 oligos. The DNA data storage archive of clause 9 or 10, wherein the data payload regions are configured to be decoded by the decoding technique.

Clause 12. The DNA data storage archive of clause 9 to 11, wherein there are at least 100 times more data storage oligonucleotides than labeling oligonucleotides.

Clause 13. This clause describes an implementation with sector 0 and sector 1 oligos. The DNA data storage archive of clause 9 to 12 further comprising, second labeling oligonucleotides comprising a decoding payload region that encodes human- or machine-readable information that comprises a second decoding technique and wherein a nucleotide sequence of the decoding payload region is decoded by the decoding technique.

Clause 14. The DNA data storage archive of clause 13, wherein the data payload regions are configured to be decoded by the second decoding technique.

Clause 15. This clause generally describes a technique for registering a new codec with the central authority. Example illustrations of this clause are in FIG. 3 and FIG. 6. A method for adding a new decoding technique (302) to a look-up table (112) comprising: receiving (602) a description of the new decoding technique and a request to add the new decoding technique to the look-up table: assigning (604) an uncoded identifier sequence (110) to the new decoding technique, wherein the uncoded identifier sequence is a sequence of nucleotides; and publishing (620) the look-up table comprising an uncoded identifier entry (114) associated with the decoding technique or with a pointer to the decoding technique.

Clause 16. The method of clause 15, wherein the new decoding technique describes at least techniques for converting nucleotide sequences to digital data while accounting for insertion, deletion, and substitution errors in the nucleotide sequences.

Clause 17. This clause generally describes implementation with only sector 0 oligos. The method of clause 15 or 16, wherein the new decoding technique describes a primer pair used for PCR amplification of data storage oligonucleotides comprising data payload regions that encode digital data.

Clause 18. The clause generally describes an uncoded identifier generated by at least a set edit distance from existing uncoded identifiers. The method of clause 15 to 17, wherein assigning the uncoded identifier sequence to the new decoding technique comprises: generating a candidate sequence of nucleotides: determining that the candidate sequence of nucleotides is sufficiently different from existing uncoded identifier entries in the look-up table; and defining the uncoded identifier sequence as the candidate sequence of nucleotides.

Clause 19. The method of clause 18, wherein determining that the candidate sequence of nucleotides is sufficiently different from existing uncoded identifiers in the look-up table comprises: calculating an edit distance between the candidate sequence of nucleotides and sequences of nucleotides of the existing uncoded identifier entries; and determining that the edit distance is greater than a threshold value.

Clause 20. This clause generally describes creating an uncoded identifier by maximizing the edit distance from existing entries in the look-up table. The method of clause 15 to 17, wherein assigning the uncoded identifier to the new decoding technique comprises identifying a sequence of nucleotides that has a maximal edit distance from uncoded identifier entries in the look-up table.

Clause 21. The method of clause 17 to 20, further comprising: determining that the candidate sequence of nucleotides has a specified property, wherein the specified property is: not containing a primer binding site for primers used for PCR amplification of labeling oligonucleotides, not containing a homopolymer run of more than three, having a GC content between about 45% and 55%, or not forming secondary structures.

Clause 22. The method of clause 15 to 21, wherein the decoding technique describes a primer pair used for PCR amplification of second labeling oligonucleotides comprising decoding payload regions that a second decoding technique.

Clause 23. The method of clause 15 to 22, wherein publishing the look-up table comprises making the look-up table available on a network-accessible database and wherein the look-up table is configured to receive queries over a network, the queries comprising a nucleotide sequence.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of +10% of the stated value.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method of identifying a decoding technique for data storage oligonucleotides that encode digital data, the method comprising:
amplifying labeling oligonucleotides in a DNA data storage archive by polymerase chain reaction (PCR) amplification using a predetermined primer pair to generate a labeling oligonucleotide amplification product;
sequencing the labeling oligonucleotide amplification product to produce an uncoded identifier sequence which is a sequence of nucleotides in the labeling oligonucleotides;
querying a look-up table with the uncoded identifier sequence; and
obtaining from the look-up table the decoding technique uniquely associated with the uncoded identifier.

2. The method of claim 1, further comprising decoding the data storage oligonucleotides in the DNA data storage archive using the decoding technique, wherein the decoding recovers the digital data from the data storage oligonucleotides.

3. The method of claim 1, further comprising:
amplifying second labeling oligonucleotides in the DNA data storage archive by PCR amplification using a second predetermined primer pair to generate a second labeling oligonucleotide amplification product;
sequencing the second labeling oligonucleotide amplification product to produce a nucleotide sequence; and
decoding the nucleotide sequence with the decoding technique to generate human- or machine-readable information that comprises a second decoding technique.

4. The method of claim 3, wherein the second predetermined primer pair is specified in the decoding technique.

5. The method of claim 3, further comprising decoding data storage oligonucleotides in the DNA data storage archive using the second decoding technique, wherein the second decoding technique recovers the digital data from the data storage oligonucleotides.

6. The method of claim 3, wherein human- or machine-readable information decoded from the second labeling oligonucleotides comprises metadata for the DNA data storage archive.

7. The method of claim 1, wherein the look-up table comprises a plurality of different decoding techniques each uniquely associated with a different uncoded identifier entry.

8. The method of claim 7, wherein querying the look-up table comprises calculating an edit distance between the uncoded identifier sequence and at least one uncoded identifier entry in the look-up table.

9. A DNA data storage archive comprising:
labeling oligonucleotides comprising an uncoded identifier sequence flanked by primer binding sites for a predetermined primer pair, wherein the uncoded identifier sequence is a sequence of nucleotides and the uncoded identifier sequence is uniquely associated in an external look-up table with a decoding technique; and
data storage oligonucleotides comprising data payload regions that encode digital data.

10. The DNA data storage archive of claim 9, wherein the primer binding sites are not found in the data storage oligonucleotides.

11. DNA data storage archive of claim 9, wherein the data payload regions are configured to be decoded by the decoding technique.

12. The DNA data storage archive of claim 9 further comprising, second labeling oligonucleotides comprising a decoding payload region that encodes human- or machine-readable information that comprises a second decoding technique and wherein a nucleotide sequence of the decoding payload region is decoded by the decoding technique.

13. The DNA data storage archive of claim 12, wherein the data payload regions are configured to be decoded by the second decoding technique.

14. A method for adding a new decoding technique to a look-up table comprising:
receiving a description of the new decoding technique and a request to add the new decoding technique to the look-up table;
assigning an uncoded identifier sequence to the new decoding technique, wherein the uncoded identifier sequence is a sequence of nucleotides; and
publishing the look-up table comprising an uncoded identifier entry associated with the decoding technique or with a pointer to the decoding technique.

15. The method of claim 14, wherein the new decoding technique describes at least techniques for converting nucleotide sequences to digital data while accounting for insertion, deletion, and substitution errors in the nucleotide sequences.

16. The method of claim 14, wherein the new decoding technique describes a primer pair used for PCR amplification of data storage oligonucleotides comprising data payload regions that encode digital data.

17. The method of claim 14, wherein assigning the uncoded identifier sequence to the new decoding technique comprises:
generating a candidate sequence of nucleotides;
determining that the candidate sequence of nucleotides is sufficiently different from existing uncoded identifier entries in the look-up table; and
defining the uncoded identifier sequence as the candidate sequence of nucleotides.

18. The method of claim 17, wherein determining that the candidate sequence of nucleotides is sufficiently different from existing uncoded identifiers in the look-up table comprises:
calculating an edit distance between the candidate sequence of nucleotides and sequences of nucleotides of the existing uncoded identifier entries; and
determining that the edit distance is greater than a threshold value.

19. The method of claim 17, further comprising:
determining that the candidate sequence of nucleotides has a specified property, wherein the specified property is:
not containing a primer binding site for primers used for PCR amplification of labeling oligonucleotides,
not containing a homopolymer run of more than three,
having a GC content between about 45% and 55%, or
not forming secondary structures.

20. The method of claim 14, wherein assigning the uncoded identifier to the new decoding technique comprises identifying a sequence of nucleotides that has a maximal edit distance from uncoded identifier entries in the look-up table.


SEQUENCE LISTING (4 sequences)

SEQ ID NO: 1 - DNA, 20 bp, synthetic construct
  source 1..20: mol_type = other DNA
tgcctagcgcctaatatggt

SEQ ID NO: 2 - DNA, 20 bp, synthetic construct
  source 1..20: mol_type = other DNA
atgtatgcggtcaggaggaa

SEQ ID NO: 3 - DNA, 20 bp, synthetic construct
  source 1..20: mol_type = other DNA
tgaccgctacgattagacca

SEQ ID NO: 4 - DNA, 20 bp, synthetic construct
  source 1..20: mol_type = other DNA
gcaaagcggttgtcttctct